(19) United States
(12) Patent Application Publication
(10) Pub. No.: US 2002/0173468 A1
(43) Pub. Date: Nov. 21, 2002

(54) MODIFIED CYTOSTATIC AGENTS

(76) Inventors: HANS-GEORG LERCHEN, LEVERKUSEN (DE); KARSTEN VON DEM BRUCH, LEVERKUSEN (DE); JORG BAUMGARTEN, WUPPERTAL (DE); MICHAEL SPERZEL, WUPPERTAL (DE)

Correspondence Address: NORRIS MCLAUGHLIN & MARCUS, P.A., 220 EAST 42ND STREET 30TH FLOOR, NEW YORK, NY, 10017, US

(21) Appl. No.: 09/269,609

(22) PCT Filed: Sep. 22, 1997
(86) PCT No.: PCT/EP97/05189

(30) Foreign Application Priority Data
Oct. 4, 1996 (DE) 196 40 970.5

(51) Int. Cl. A61K 38/00; C07K 5/00; C07K 7/00; C07K 16/00; C07K 17/00; A61K 38/04

(52) U.S. Cl. 514/17; 530/329; 530/330

(57) ABSTRACT

The present invention relates to conjugates of cytostatics and N-thiocarbonyl-modified amino acids or peptides, processes for their preparation and their use as medicaments, in particular in connection with carcinomatous disorders.

The present invention relates to conjugates of cytostatics and N-thiocarbonyl-modified amino acids or peptides, processes for their preparation and their use as medicaments, in particular in connection with carcinomatous disorders.

Chemotherapy in carcinoses is accompanied by side effects which are usually serious, caused by the toxicity of chemotherapeutics on proliferating cells of other tissues. For many years, scientists have occupied themselves with the problem of improving the selectivity of active compounds employed. An approach which is frequently followed is the synthesis of prodrugs, which are released to a more or less selective extent in the target tissue, for example, by changing the pH (e.g. Tietze et al., DE 4 229 903), by enzymes (e.g. glucuronidases; Jacquesy et al., EP 511 917; Bosslet et al., EP 595 133) or by antibody-enzyme conjugates (Bagshawe et al., WO 88/07378; Senter et al., U.S. Pat. No. 4,975,278; Bosslet et al., EP 595 133). A problem in these approaches is, inter alia, the lack of stability of the conjugates in other tissues and organs and in particular the ubiquitous active compound distribution which follows the extracellular release of active compound in the tumour tissue.

Below, three cytostatically active parent substances from various substance classes which are affected by serious side effects are presented by way of example.

The heterocyclic amine batracylin (1) shows a good antitumour action in various intestinal cancer models (U.S. Pat. No. 4 757 072).

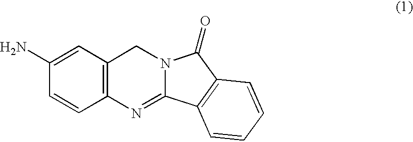

Peptide conjugates of (1) having good in-vitro action and more favourable solubility properties (U.S. Pat. No. 4,180,343) have a worse tolerability in animal experiments than batracylin itself. Thus, for example, the fucose conjugates described in EP 501 250 concentrate very strongly in the liver. Glycoconjugates of cytostatics, such as are described in our likewise pending application PCT/96/01279, do have more favourable properties, but are accessible synthetically only with relatively great expenditure.

In addition to an outstanding antibacterial activity, quinolone-a (2) 7-[(3aRS, 4RS, 7aSR)-4-amino-1,3,3a,4,7,7a-hexahydro-iso-indol-2-yl]-8-chloro-1-cyclopropyl-6-fluoro-1,4-dihydro-4-oxo-3-quinolinecarboxylic acid also shows a very good activity against various tumour cell lines (EP 520 240, JP 4 253 973). However, substantial toxicological problems face it (e.g. genotoxicity, bone marrow toxicity, high acute toxicity in vivo etc.).

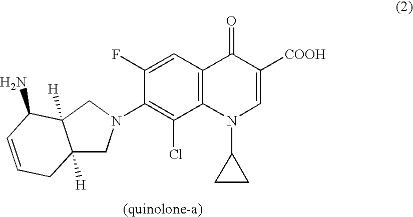

20(S)-Camptothecin (3) is a pentacyclic alkaloid which was isolated by Wall et al., (J. Amer. Chem. Soc. 88 (1966) 3888). It has a high antitumour active potential in numerous in-vitro and in-vivo tests. Unfortunately, however, the realization of the very promising potential fails in the clinic because of toxicity and solubility problems.

By opening the E-ring lactone and formation of the sodium salt, a water-soluble compound was obtained which is in a pH-dependent equilibrium with the ring-closed form. Up until now, clinical studies here also have not led to success.

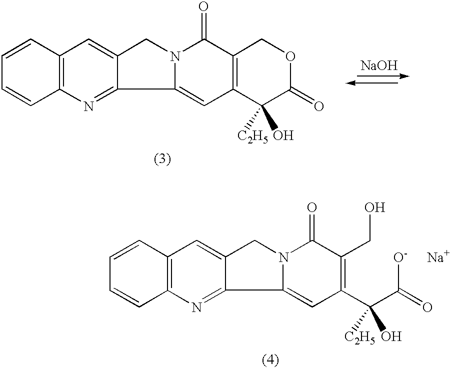

About 20 years later, it was found that the biological activity can be attributed to enzyme inhibition of the topoisomerase I. Since then, the research activities have been increased again in order to find camptothecin derivatives which are more tolerable and active in vivo.

To improve the water solubility, for example, salts of A ring- and B ring-modified camptothecin derivatives and of 20-O-acyl derivatives having ionizable groups have been described (Vishnuvajjala et al., U.S. Pat. No. 4,943,579). The latter prodrug concept was later transferred to modified camptothecin derivatives (Wani et al., WO 96/02546). The 20-O-acyl prodrugs described, however, have a very short half-life in vivo and are very rapidly cleaved to give the parent substance.

We have now found that the modification of cytostatics such as, for example, batracylin, antitumour-active quinolones (such as, for example quinolone-a) or camptothecin and camptothecin derivatives with N-thiocarbonyl-modified amino acids leads to new compounds having surprising, highly interesting properties:

The conjugates thus obtained are easily accessible synthetically and show a similarly high activity in vitro to various tumour cell lines and tumour xenografts as the underlying toxophore.

Depending on the composition of the N-thiocarbonyl-modified amino acids, the conjugates according to the invention show significantly improved solubility properties in comparison with the underlying cytostatics.

Compared with the underlying toxophores, they have a higher tolerability and tumour selectivity.

In vivo, they show a good to very good therapeutic activity.

In extracellular medium and in blood, they are significantly more stable than the previously described pure amino acid prodrugs of batracylin, quinolones or of camptothecin derivatives.

In the case of 20-O-acylations of camptothecin derivatives, the lactone ring important for the activity is stabilized by the ester-like linkage of the carrier radicals with the 20-hydroxy group.

The invention relates to compounds of the general formula (I)

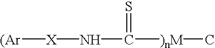

in which

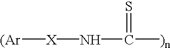

represents 1 to n′ groups

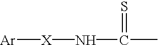

which are identical to or different from one another, where n is a number 1 to n′ and n′ corresponds to the maximum number of possible linkage sites of M,

in which

Ar represents an aryl radical having up to 10 carbon atoms, which additionally to X can optionally be mono- or polysubstituted by alkyl having up to 6 carbon atoms, alkoxy having up to 6 carbon atoms, alkoxycarbonyl having up to 6 carbon atoms, hydroxyl, carboxyl, carboxyalkyl having up to 6 carbon atoms, cyano, nitro, isocyanato, isothiocyanato, halogen, sulphonyl and/or sulphonamide,

X represents a direct single bond or alkylene having up to 6 carbon atoms,

M represents a mono-, di-, tri- or tetrapeptide, which is linked via the α-amino group and/or via amino and/or hydroxy groups of the side chains to the n groups

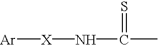

which are identical to or different from one another, where further functional groups of the peptide can optionally carry protective groups,

C represents a radical of a cytostatic or of a cytostatic derivative which is linked to M via an amino function or via an oxygen atom,

and their stereoisomers, stereoisomer mixtures and salts.

C can be an intercalating substance, a topoisomerase inhibitor, an antimetabolite, an alkylating agent, a tubulin inhibitor, a tyrosine phosphokinase inhibitor, a protein kinase C inhibitor or an active compound having another or unknown cytostatic or cytotoxic mechanism of action. C can be, for example, a nucleoside, an enediine antibiotic, a quinolone- or naphthyridonecarboxylic acid or a cytotoxic peptide antibiotic, e.g. from the dolastatins class. C can be batracylin, quinolone-a, 5-fluorouracil, cytosine arabinoside, methotrexate, etoposide, camptothecin, a camptothecin derivative, daunomycin, doxorubicin, taxol, vinblastine, vincristine, dynemicin, calicheamycin, esperamycin, quercetin, suramin, erbstatin, cyclophosphamide, mitomycin C, melphalan, cisplatin, bleomycin, staurosporin or another active compound having antineoplastic activity.

The term “alkyl groups” is intended here, if not stated otherwise, to include alkyl radicals containing straight-chain, branched, cyclic and cycloalkyl radicals. This definition is correspondingly also intended to apply to all other radicals containing alkyl groups, such as, for example, alkoxy etc.

Preferred compounds of the formula (I) are those in which

Ar represents a phenyl radical which can additionally carry hydroxyl, carboxyl, isothiocyanato or halogen in the para-position to X.

Additionally preferred compounds of the formula (I) are those in which

X represents a single bond or methylene.

Additionally preferred compounds of the formula (I) are those

in which

M represents a mono-, di- or tripeptide which is linked to the n

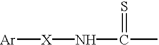

groups which are identical to or different from one another via the α-amino group and/or via amino and/or hydroxy groups of the side chains, where further functional groups of the peptide can optionally carry protective groups.

Preferably, the peptides M consist of amino acid radicals which are derived from alanine, aspartic acid, glutamic acid, glycine, leucine, histidine, lysine, arginine, ornithine, serine, tyrosine, valine, diaminopropionic acid, α, γdiaminobutyric acid or phenylalanine, it being possible for a number of amino acid radicals to be linked in peptide form both via the α-amino group and optionally via the side chain amino functions or alternatively via both functions.

If M carrier further functional groups, these are preferably deblocked.

Additionally preferred compounds of the formula (I) are those in which C represents a batracylin, methotrexate, quinolone-a, etoposide, melphalan, taxol or camptothecin radical, a camptothecin derivative modified in the A ring or B ring, a daunomycin or doxorubicin radical, where C is linked to M via an amino or hydroxyl function. Very particularly preferred examples of C are radicals of batracylin, quinolone-a and doxorubicin, camptothecin, 7-ethylcamptothecin; 10,11-(methylenedioxy)-camptothecin; 7-hydroxymethylcamptothecin and 7-ethyl-10-hydroxycamptothecin.

The compounds according to the invention can be present in stereoisomeric forms, for example as enantiomers or diastereomers, or as their mixtures, for example as a racemate. The invention relates both to the pure stereoisomers and to their mixtures.

If necessary, the stereoisomer mixtures can be separated into the stereoisomerically homogeneous constituents in a known manner, for example by chromatography or by crystallization processes.

The amino acid radicals can each be present in the D form or in the L form.

The nomenclature of the amino acids follows the rules drawn up by the IUPAC. If indication of the stereochemistry is missing, amino acids of the L form were employed.

As result of prevention of rotation, the compounds according to the invention can occur in rotational isomer forms or as their mixtures. The invention relates both to the pure rotational isomers and their mixtures.

Rotational isomer mixtures can optionally be separated into the homogeneous constituents, if necessary, by means of known methods, for example by chromatography (e.g. HPLC) or by crystallization processes. This is possible not only at the final stage of the conjugates, but optionally also at intermediate stages.

The rotamerically pure final substances can be prepared from the rotamerically pure intermediates, if appropriate by a suitable synthetic procedure.

The compounds according to the invention can also be present in the form of their salts. In general, salts with organic or inorganic bases or acids and also internal salts may be mentioned here.

The acids which can be added preferably include hydrohalic acids, such as, for example, hydrochloric acid and hydrobromic acid, in particular hydrochloric acid, furthermore phosphoric acid, nitric acid, sulphuric acid, mono- and bifunctional carboxylic acids and hydroxycarboxylic acids, such as, for example, acetic acid, trifluoroacetic acid, maleic acid, malonic acid, oxalic acid, gluconic acid, succinic acid, fumaric acid, tartaric acid, citric acid, salicylic acid, sorbic acid and lactic acid as well as sulphonic acids, such as, for example, p-toluenesulphonic acid, 1,5-naphthalenedisulphonic acid or camphorsulphonic acid.

Physiologically acceptable salts can also be metal or ammonium salts of those compounds according to the invention which have a free carboxyl group. Those particularly preferred are, for example, sodium, potassium, magnesium or calcium salts, and also ammonium salts which are derived from ammonia or organic amines such as, for example, ethylamine, di- or triethylamine, di- or triethanolamine, dicyclohexylamine, dimethylaminoethanol, arginine, lysine, ethylenediamine or phenethylamine.

The invention furthermore relates to a process for the preparation of compounds of the general formula (I), characterized in that compounds of the general formula (II)

M′—C (II),

in which C has the meaning indicated above and M′ represents a radical M which carries hydrogen atoms on the desired linkage sites and whose other potential linkage sites are blocked by protective groups,

are reacted with compounds of the general formula (III)

Ar—X—N═C═S (III)

in suitable solvents in the presence of a base to give compounds of the general formula (Ia)

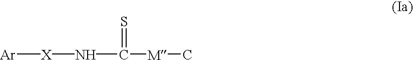

in which Ar, X and C have the meanings indicated above and M″ represents a radical M, whose further potential linkage sites are blocked by protective groups,

and in the case of the introduction of further groups

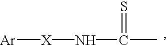

which differ from that or those initially introduced, the corresponding protective groups are optionally selectively removed from the compounds of the formula (Ia), the latter are reacted in the manner indicated above with further compounds of the general formula (III), which differ from those initially introduced, and, if appropriate, this reaction sequence is repeated to introduce further radicals

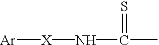

different from the radicals introduced,

and in that remaining protective groups are optionally removed.

The conjugates according to the invention can be prepared, for example, by linkage of cytostatic derivatives carrying hydroxy or amino groups (e.g. batracylin, quinolones or camptothecins) with activated carboxyl components which, for their part, can be parts of protected amino acids, peptides or N-thiocarbonyl-modified peptides.

The compounds of the general formula (II) are accessible by linking optionally protected amino acid units to amino or hydroxy functions of C by customary methods of peptide chemistry and, if appropriate, constructing a peptide chain by stepwise introduction of further amino acid units. Alternatively, peptide units optionally carrying protective groups can also be linked to C according to customary methods.

The reactions can be carried out under various pressure and temperature conditions, for example 0.5 to 2 bar, and −30 to +100° C., in suitable solvents such as dimethylformamide (DMF), tetrahydrofuran (THF), dichloromethane, chloroform, lower alcohols, acetonitrile, dioxane, water or in mixtures of the solvents mentioned. As a rule, reactions in DMF or THF/dichloromethane at normal pressure and at a temperature of 0 to 60° C., in particular at approximately room temperature, are preferred.

For the activation of the carboxyl groups, suitable coupling reagents are those known in peptide chemistry such as described, for example, in Jakubke/Jeschkeit: Aminosäuren, Peptide, Proteine [Amino Acids, Peptides, Proteins]; Verlag Chemie 1982 or Tetrahedr. Lett. 34, 6705 (1993). For example, acid chlorides, N-carboxylic anhydrides or mixed anhydrides are preferred.

Additionally preferred for the activation of the carboxyl groups is the formation of adducts with carbodiimides, e.g. N,N′-diethyl-, N,N′-diisopropyl-, N,N′-dicyclohexylcarbodiimide, N-(3-dimethylaminopropyl)-N′-ethyl-carbodiimide hydrochloride, N-cyclohexyl-N′-(2-morpholinoethyl)-carbodiimide metho-p-toluenesulphonate, or carbonyl compounds such as carbonyldiimidazole, or 1,2-oxazolium compounds such as 2-ethyl-5-phenyl-1,2-oxazolium-3-sulphate or 2-tert-butyl-5-methyl-isoxazolium perchlorate, or acylamino compounds such as 2-ethoxy-1-ethoxycarbonyl-1,2-dihydroquinoline, or propanephosphonic anhydride, or isobutyl chloroform, or benzotriazolyloxy-tris(dimethylamino)phosphonium hexafluorophosphate, 1-hydroxybenzotriazole or hydroxysuccinimide esters.

Bases which can be employed are, for example, triethylamine, Hünig's base, ethyldiisopropylamine, pyridine, N,N-dimethylaminopyridine or others.

Protective groups which can be employed for possible further reactive functions in the cytostatic part or for ternary functions of the amino acids are the protective groups known in peptide chemistry, for example of the urethane, alkyl, acyl, ester or amide type.

Amino protective groups in the context of the invention are the customary amino protective groups used in peptide chemistry.

These preferably include: benzyloxycarbonyl, (Cbz) 3,4-dimethoxybenzyloxycarbonyl, 3,5-dimethoxybenzyloxycarbonyl, 2,4-dimethoxybenzyloxycarbonyl, 4-methoxybenzyloxycarbonyl, 4-nitrobenzyloxycarbonyl, 2-nitrobenzyloxycarbonyl, 2-nitro-4,5-dimethoxybenzyloxycarbonyl, methoxycarbonyl, ethoxycarbonyl, tert-butoxycarbonyl, (Boc)allyloxycarbonyl, vinyloxycarbonyl, 3,4,5-tri-methoxybenzyloxycarbonyl, phthaloyl, 2,2,2-trichloroethoxycarbonyl, 2,2,2-tri-chloro-tert-butoxycarbonyl, methoxyloxycarbonyl, 4-nitrophenoxycarbonyl, fluorenyl-9-methoxycarbonyl(Fmoc), formyl, acetyl, propionyl, pivaloyl, 2-chloroacetyl, 2-bromoacetyl, 2,2,2-trifluoroacetyl, 2,2,2-trichloroacetyl, benzoyl, benzyl, 4-chlorobenzoyl, 4-bromobenzoyl, 4-nitrobenzoyl, phthalimido, isovaleroyl or benzyloxymethylene, 4-nitrobenzyl, 2,4-dinitrobenzyl, 4-nitrophenyl or 2-nitro-phenylsulphenyl. Particularly preferred protective groups are Fmoc, Boc and Cbz.

The protective groups in the corresponding reaction steps can be removed, for example, by the action of acid or base, hydrogenolytically or reductively in another manner.

### Biological Testing

1. Growth Inhibition Test for the Determination of Cytotoxic Properties

The human colonic tumour cell lines SW 480 and HT 29 (ATCC No. CCL 228 and HBT-38) and the mouse melanoma cell line B16F10 were grown in Roux dishes in RPMI 1640 medium with addition of 10% FCS. They were then trypsinized and taken up in RPMI plus 10% FCS to give a cell count of 50,000 cells/ml. 100 μl of cell suspension/well were added to a 96 microwell plate and incubated for 1 day at 37° C. in a CO₂ incubator. A further 100 μl of RPMI medium and 1 μl of DMSO were then added with the test substances. The growth was checked after day 3 and day 6. To do this, 40 μl of MTT solution (3-(4,5-dimethylthiazol-2-yl)-2,5-diphenyl-tetrazolin bromide) were added to each microwell at a starting concentration of 5 mg/ml of H₂O. Incubation was carried out at 37° C. for 5 hours in the CO₂ incubator. The medium was then aspirated and 100 μl of i-propanol/well were added. After shaking for 30 min with 100 μl of H₂O, the extinction was measured at 540 nm using a Titertek Multiskan MCC/340 (flow).

The cytotoxic action is indicated in Table 1 as an IC₅₀ value in each case for the SW 480 and HT 29 and B16F10 cell lines:

TABLE 1

| | IC₅₀/μM | IC₅₀/μM | IC₅₀/μM |
|---|---|---|---|
| Example | SW 480 | HT 29 | B16F10 |
| 1.2) | 25 | 40 | — |
| 1.3) | 45 | 70 | — |
| 1.4) | 40 | 40 | 30 |
| 1.5) | 250 | 400 | — |
| 1.6) | 550 | 800 | — |
| 2.1) | 20 | 9 | 9 |
| 2.2) | 15 | 6 | 4 |
| 2.3) | 0.9 | 0.7 | 0.2 |
| 2.4) | 0.8 | 0.9 | 1 |
| 2.5) | 200 | >200 | 200 |
| 2.6) | 0.2 | 0.3 | 0.06 |
| 2.8) | 0.2 | 0.1 | 0.1 |
| 2.9) | 2 | 2 | 1 |
| 2.10) | 2 | 2 | 0.4 |
| 2.11) | 60 | 150 | 30 |
| 3) | 3 | 2 | 1 |
| 4.1) | 0.01 | 0.02 | 0.1 |
| 4.2) | 0.07 | 0.06 | 0.3 |
| 4.3) | 0.02 | 0.02 | 0.1 |
| 4.4) | 0.3 | 0.2 | 0.6 |
| 4.5) | 0.3 | 0.2 | 0.8 |
| 4.6) | 0.2 | 0.15 | 0.5 |
| 4.7) | 0.1 | 0.06 | 0.3 |
| 4.8) | 0.3 | 0.15 | 0.8 |
| 4.9) | 0.02 | 0.015 | 0.2 |
| 4.10) | 0.02 | 0.01 | 0.2 |
| 4.11) | 0.06 | 0.03 | 0.2 |
| 4.12) | 0.04 | 0.03 | 0.2 |
| 4.13) | 0.06 | 0.04 | 0.2 |
| 4.14) | 0.16 | 0.075 | 0.75 |
| 4.15) | 0.09 | 0.06 | 0.2 |
| 4.16) | 0.15 | 0.12 | 0.6 |
| 4.17) | 0.3 | 0.17 | 0.8 |
| 4.18) | 0.3 | 0.12 | 0.4 |
| 4.19) | 0.08 | 0.04 | 0.4 |
| 4.20) | 0.07 | 0.06 | 0.3 |
| 4.21) | 0.7 | 0.3 | 3 |
| 4.22) | 0.04 | 0.04 | 0.1 |
| 4.23) | 0.08 | 0.07 | 0.15 |
| 5.1) | 0.025 | 0.02 | 0.05 |
| 5.2) | 0.5 | 0.3 | 0.9 |
| 6) | 0.005 | 0.003 | 0.015 |
| 7) | 0.06 | 0.08 | 1 |
| 8) | 0.15 | 0.2 | 3.0 |

2. Haematopoietic Activity of Conjugates in Comparison with the Basic Active Compound

Material and Methods

Bone marrow cells were rinsed from mouse femurs. 10⁵ cells were incubated in McCoy 5A medium (0.3% agar) together with recombinant murine GM-CSF (Genzyme; stem cell colony formation) and the substances (10⁻⁴ to 100 μg/ml) at 37° C. and 7% CO₂. 7 days later, the colonies (<50 cells) and clusters (17-50 cells) were counted.

Results

As presented in Table 2, the conjugates investigated show a drastically decreased inhibition of bone marrow stem cell proliferation compared with the basic active compound.

TABLE 2
Inhibition of the CSF-induced proliferation of mouse
bone marrow stem cells

| Example | IC₅₀ [ng/ml] |
|---|---|
| Quinolone-a | 0.2 |
| 2.4) | 60.0 |
| Camptothecin | 0.4 |
| 4.4) | 10 |
| 4.9) | 22 |

3. In-vivo Inhibition of Tumour Growth in the Nude Mouse Model

Material

For all in-vivo experiments for investigation of the inhibition of tumour growth, athymic nude mice (NMRI nu/nu strain) were used. The selected large-cell lung carcinoma LXFL 529 was developed by serial passage in nude mice. The human origin of the tumour was confirmed by isoenzymatic and immunohistochemical methods.

Experimental Set-up

The tumour was implanted subcutaneously in both flanks of nu/nu nude mice 6 to 8 weeks old. The treatment was started, depending on the doubling time, as soon as the tumours had reached a diameter of 5-7 mm. The mice were assigned to the treatment group and to the control group (5 mice per group with 8-10 assessable tumours) by randomization. The individual tumours of the control group all grew progressively.

The size of the tumours was measured in two dimensions by means of a slide gauge. The tumour volume, which correlated well with the cell count, was then used for all evaluations. The volume was calculated according to the formula “length×breadth×breadth/2” ([a×b²]/2, a and b represent two diameters arranged at right angles).

The values of the relative tumour volume (RTV) were calculated for each individual tumour by dividing the tumour size on day X by the tumour size on day 0 (at the time of randomization). The mean values of the RTV were then used for further evaluation.

The inhibition of the increase in the tumour volume (tumour volume of the test group/control group, T/C, in per cent) was the final measured value.

Treatment

The administration of the compounds was carried out intraperitoneally (i.p.) on days 1, 2 and 3 after randomization.

Results

The therapeutic efficacy of the conjugates according to the invention compared with the large-cell human lung tumour xenograft LXFL 529 is represented with the aid of the compound from Example 4.4). Therapy at the maximum tolerable dose (MTD) and at half the MTD leads to tumour remission.

TABLE 3

| | | | | Relative | Relative |
|---|---|---|---|---|---|
| | Dose | Survival | Number of | tumour volume | body weight |
| | [mg/kg/ | time | tumours | on day 21 | on day 21 |
| Therapy | day] | [days] | [day 21] | [% of day 0] | [% of day 0] |
| Control | — | >39 35 | 16 | 1137 | 111.6 |
| group | | 35 >18 | | | |
| | | >35 | | | |
| Example | 6.25 | 7 >43 | 9 | 0.2 | 113.0 |
| 4.4) | (MTD) | >43 >43 | | | |
| | | >43 | | | |
| Example | 3.125 | >43 >43 | 7 | 69.5 | 105.8 |
| 4.4) | | >43 >43 | | | |

The compounds according to the invention have a surprisingly strong cytotoxic activity both in vitro and in vivo against various tumours, in particular those of the lung and of the large intestine, combined with a great selectivity towards non-malignant cells.

They are therefore suitable for the treatment of carcinomatous disorders, in particular of those of the lung and of the large intestine.

The present invention includes pharmaceutical preparations which, in addition to non-toxic, inert pharmaceutically suitable excipients, contain one or more compounds according to the invention or which consist of one or more active compounds according to the invention, and to processes for the production of these preparations.

The active compound(s) can optionally also be present in microencapsulated form in one or more of the excipients indicated above.

The therapeutically active compounds should be present in the abovementioned pharmaceutical preparations preferably in a concentration of approximately 0.1 to 99.5%, preferably of approximately 0.5 to 95%, by weight of the total mixture.

Apart from the compounds according to the invention, the abovementioned pharmaceutical preparations can also contain further pharmaceutical active compounds.

In general, it has proved advantageous both in human and in veterinary medicine to administer the active compound(s) according to the invention in total amounts of approximately 0.5 to approximately 500, preferably 5 to 100, mg/kg of body weight every 24 hours, if appropriate in the form of several individual doses, to achieve the desired results. An individual dose contains the active compound(s) according to the invention preferably in amounts from approximately 1 to approximately 80, in particular 3 to 30, mg/kg of body weight.

### Synthesis Examples

All thiocarbonyl-amino acid or thiocarbonyl-peptide conjugates which are the subject of this invention are synthesized according to the following general procedure:

A solution of 1 mmol of the basic amino acid or peptide conjugate in 50 ml of absolute dimethylformamide is treated with 1.1 mmol each of the appropriate isothiocyanate per free amino group. After addition of 1.74 ml (10 mmol) of ethyldiisopropylamine, the mixture is stirred at room temperature until amino acid or peptide conjugate can no longer be detected in the thin-layer chromatogram, but at the longest for 16 h. The mixture is concentrated in vacuo and the residue is purified, after drying in high vacuum, by flash chromatography on silica gel, e.g. using an ethyl acetate/petroleum ether or a dichloromethane/methanol system. Reprecipitation a number of times from dichloromethane/methanol 1:1 (v/v) with diethyl ether frequently also affords pure products.

Residual protective groups are then removed in a second stage by processes known from the literature (a fluorenyl-9-methoxycarbonyl group, for example, using piperidine in absolute dimethylformamide at room temperature; a tert-butoxycarbonyl group, for example, using trifluoroacetic acid in absolute dichloromethane at room temperature).

The appropriate isothiocyanates can be acquired in the specialist chemicals business or are synthesized by methods known from the literature.

### Synthesis Examples of Precursors: Amino Acid and Peptide Conjugates

### EXAMPLE I.1

### N-[N^{ε}-(Fluorenyl-9-methoxycarbonyl)-lysyl]-batracylin, trifluoroacetate

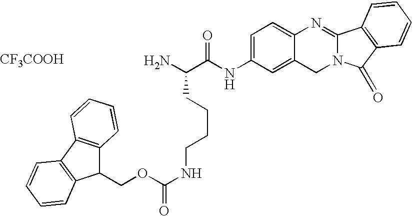

I.1.a) N-[N^{α}-(tert-Butoxycarbonyl)-N^{ε}-(fluorenyl-9-methoxycarbonyl)-lysyl]-batracylin

N^{α}-(tert-Butoxycarbonyl)-N^{ε}-(fluorenyl-9-methoxycarbonyl)-lysine (5.3 g, 11.3 mmol) and 2-isobutoxy-1-isobutoxycarbonyl-1,2-dihydro-quinoline (4 ml, 14 mmol) are dissolved in 40 ml of dichloromethane. After stirring at room temperature for 20 min, a solution of batracylin (2.5 g, 10 mmol) in dimethylformamide (80 ml) is added and the mixture is stirred at room temperature for a further 24 h. It is then concentrated in vacuo until crystallization commences. The suspension obtained is treated with ethanol (500 ml) and refluxed for 1 h. After cooling to room temperature, the product is filtered off and washed with acetone and then with diethyl ether. Yellow crystals (5.9 g, 84%) are obtained [TLC (ethyl acetate): R_{f}=0.57; m.p.=158° C. (dec.)].

I.1) N-[N⁶⁸-(Fluorenyl-9-methoxycarbonyl)-lysyl]-batracylin, trifluoroacetate

A suspension of the above compound (5.6 g, 8 mmol) in dichloromethane (75 ml) is treated with anhydrous trifluoroacetic acid (25 ml) and the resulting solution is stirred at room temperature for 90 min. After concentrating in vacuo, the residue is crystallized by addition of diethyl ether (200 ml). The precipitate is filtered off and washed intensively with diethyl ether. After reprecipitating a number of times from dichloromethane/methanol 1:1 using diethyl ether, yellow-orange crystals (5.13 g, 90%) are obtained [TLC (ethyl acetate): Rf=0.05; m.p.=162 ° C. (dec.)].

### EXAMPLE I.2

### N-[Seryl-D-alanyl]-batracylin, trifluoroacetate

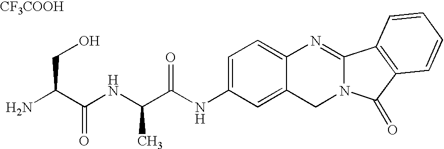

I.2.a) N-[N-Benzyloxycarbonyl-D-alanyl]-batracylin

N-Benzyloxycarbonyl-D-alanine (3.9 g, 17.5 mmol) is reacted with batracylin (4.1 g, 16.4 mmol) in analogy to Example I.1.a. After concentrating in vacuo to 50 ml, the residue is made up to 300 ml with ethyl acetate and immediately heated to boiling for 10 min. It is then allowed to cool to room temperature, filtered off and the filtered material is extracted by boiling again with ethyl acetate (200 ml). Cooling with stirring to 0° C. and filtration affords yellow crystals. The crystals (6.4 g, 80%) are removed by filtration and the combined filtrates are purified by flash chromatography [petroleum ether/ethyl acetate 3:2→1:1] after concentrating in vacuo. A further 1.35 g (17%) are obtained [TLC (ethyl acetate): R_{f}=0.45; m.p.=256° C.; [α]²⁰=+75.1° (c=1.0/CH₂Cl₂+0.5% CH₃OH)].

I.2.b) N-[D-Alanyl]-batracylin

Compound I.2.a (11.4 g, 25 mmol) is dissolved in a 33% strength solution of hydrogen bromide in glacial acetic acid (100 ml). After 30 min at room temperature, the mixture is concentrated to 30 ml in vacuo and then poured into saturated sodium hydrogencarbonate solution (1000 ml) with vigorous stirring. Stirring is continued for 10 min, and the solid is filtered off and washed with water, a little isopropanol and diethyl ether. The product is obtained in yellow crystals (7.87 g, 98%) [TLC (ethyl acetate): R_{f}=0.06; m.p.=267° C. (dec.)].

I.2.c) N-[N-(tert-Butoxycarbonyl)-seryl-D-alanyl]-batracylin

Preparation in analogy to Example I.1.a from N-(tert-butoxycarbonyl)-serine and N-[D-alanyl]-batracylin (Example I.2.b); yield: 77%.

I.2) N-[Seryl-D-alanyl]-batracylin, trifluoroacetate

Preparation in analogy to Example I.1 from compound I.2.c; yield: 98%.

### EXAMPLE I.3

### N-[N^{ε}-(Fluorenyl-9-methoxycarbonyl)-lysyl-D-alanyl]-quinolone-a, trifluoroacetate

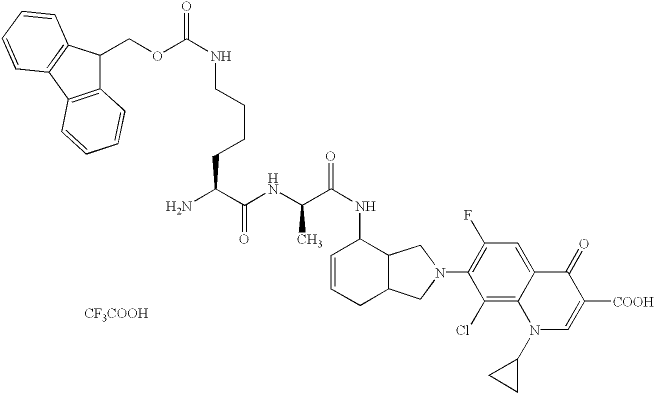

I.3.a) N-[N-(tert-Butoxycarbonyl)-D-alanyl]-quinolone-a

N-(tert-Butoxycarbonyl)-D-alanine (3.6 g, 19.2 mmol) and 2-isobutoxy-1-isobutoxy-carbonyl-1,2-dihydro-quinoline (5.8 g, 19.2 mmol) are dissolved in 200 ml of dimethylformamide. After stirring at room temperature for 8 h, quinolone-a (4 g, 9.6 mmol) and ethyldiisopropylamine (3.3 ml) are added and the mixture is treated with ultrasound for 10 h. It is concentrated, the residue is taken up in dichloromethane and the mixture is precipitated with ether. After filtration, washing with ether and drying in a high vacuum, 4.58 g (81%) of the target product are obtained, which is reacted without further purification.

I.3.b) N-[D-Alanyl]-quinolone-a, trifluoroacetate

4.56 g (7.75 mmol) of the compound from the above example are dissolved in a mixture of dichloromethane (50 ml) and anhydrous trifluoroacetic acid (50 ml) at 0° C. and stirred at this temperature for 1 h. The mixture is concentrated, redistilled with dichloromethane and the residue is reprecipitated from methanol using diethyl ether. 4.07 g (87%) of the crystalline target product are obtained [TLC (aceto-nitrile/water/glacial acetic acid 5:1:0.2): R_{f}=0.34].

I.3.c) N-[N^{α}-(tert-Butoxycarbonyl)-N^{ε}-(fluorenyl-9-methoxycarbonyl)-lysyl-D-alanyl]-quinolone-a

N^{α}-tert-Butoxycarbonyl)-N^{ε}-(fluorenyl-9-methoxycarbonyl)-lysine (1.57 g, 3.36 mmol) is dissolved in dimethylformamide (25 ml) and treated at 0° C. with N-hydroxysuccinimide (600 mg, 5.04 mmol) and N,N′-dicyclohexylcarbodiimide (820 mg, 4.03 mmol). After 3 h, the resulting urea is filtered off, 1.5 g (2.86 mmol) of the compound from Example I.3.b) are added to the filtrate and it is stirred at room temperature for 16 h. Residual urea is filtered off and the filtrate is purified by flash chromatography [dichloromethane/methanol 97.5:2.5→90:10]. The mixture is then reprecipitated from dichloromethane/methanol 1:1 using diethyl ether. Yield: 1.5 g (56%) [TLC (dichloromethane/methanol 9:1): R_{f}=0.47].

I1.3) N-[N^{ε}-(Fluorenyl-9-methoxyearbonyl)-lysyl-D-alanyl]-quinolone-a, trifluoroacetate

Removal of the tert-butoxycarbonyl group from compound I.3.c in analogy to Example I.1 and reprecipitation of the crude product from methanol with diethyl ether affords yellow crystals. Yield: 80% [TLC (dichloromethane/methanol/ammonia (17% strength) 15:4:0.5): R_{f}=0.36].

### EXAMPLE I.4

### 20-O-[N^{ε}-(Fluorenyl-9-methoxycarbonyl)-lysyl-alanyl]-camptothecin, trifluoroacetate

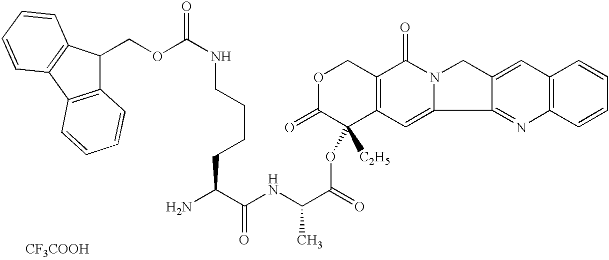

I.4.a) 20-O-(Alanyl)-camptothecin, trifluoroacetate

Camptothecin (500 mg, 1.44 mmol) is dissolved in absolute dimethylformamide (20 ml) and then treated with 4-dimethylaminopyridine (50 mg) and N-tert-butoxy-carbonyl-alanine-N-carboxy-anhydride (775 mg, 3.6 mmol). After 3 h, a further 775 mg (3.6 mmol) of N-tert-butoxycarbonyl-alanine-N-carboxy-anhydride are added and the suspension is treated with ultrasound for 16 h. It is concentrated, the crude material is taken up in dichloromethane (50 ml) and 5 ml of trifluoroacetic acid are added at 0° C. After stirring for 30 min, the mixture is concentrated again and the product is purified by flash chromatography (acetonitrile/water 20:1). The appropriate fractions are collected, concentrated and, after dissolving in dioxane/water, lyophilized. 712 mg (93%) of the target compound are obtained [FAB-MS: m/e=420 (M+H)⁺].

I.4) 20-O-[N^{ε}-(Fluorenyl-9-methoxycarbonyl)-lysyl-alanyl]-camptothecin, trifluoroacetate

The conjugate from Example I.4.a is linked to N^{α}-(tert-butoxycarbonyl)-N^{ε}-(fluorenyl-9-methoxycarbonyl)-lysine according to the standard procedure (see Example I.1.a) and then deblocked on the α-amino function in analogy to Example I.1. The target compound is obtained in a yield of 24% [TLC (aceto-nitrile/water 20:1): R_{f}=0.15].

### EXAMPLE I.5

### 7-Ethyl-20-O-(lysyl-alanyl)-camptothecin, di-trifluoroacetate

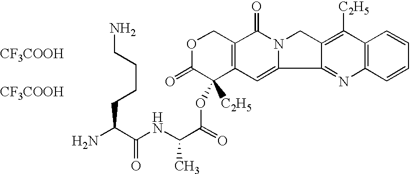

I.5.a) 7-Ethyl-20-O-[N-(tert-butoxycarbonyl)-alanyl]-camptothecin

A solution of 1.88 g (5.0 mmol) of 20(S)-7-ethyl-carnptothecin (S. Sawada et al., Chem.Pharm.Bull. 39 (1991) 1446-1454) in 100 ml of absolute dimethylformamide is treated with stirring with 2.15 g (10.0 mmol) of N-(tert-butoxycarbonyl)-alanine-N-carboxylic anhydride and 150 mg (1.2 mmol) of 4-(N,N-dimethylamino)-pyridine. After 3 h at room temperature, a further 2.15 g (10.0 mmol) of N-(tert-butoxy-carbonyl)-alanine-N-carboxylic anhydride and 150 mg (1.2 mmol) of 4-(N,N-di-methylamino)-pyridine are added and the mixture is stirred overnight at room temperature. It is then concentrated in vacuo and the residue is purified by flash chromatography [petroleum ether/ethyl acetate 2:1→1:1→ethyl acetate]. 2.02 g (73.8%) of colourless crystals are obtained [TLC (ethyl acetate): R_{f}=0.56; m.p.=206-212° C.; FAB-MS: m/e=548 (M+H⁺)].

I.5.b) 20-O-Alanyl-7-ethyl-camptothecin, trifluoroacetate

A solution of compound I.5.a (1.81 g, 3.3 mmol) in a mixture of 70 ml of dichloromethane and 7 ml of anhydrous trifluoroacetic acid is stirred at room temperature for 90 min. After concentrating in vacuo to a small volume, the product is precipitated with diethyl ether and washed thoroughly with diethyl ether. 1.34 g (72.3%) of pale yellow crystals are obtained [TLC (ethyl acetate): R_{f}=0.05; m.p.=242° C. (dec.)].

I.5.c) 7-Ethyl-20-O-[N^{α},N^{ε}-di-(tert-butoxycarbonyl)-lysyl-alanyl]-camptothecin

1.57 g (4.55 mmol) of N,N-Di-(tert-butoxycarbonyl)-lysine and 923 mg (6.83 mmol) of 1-hydroxy-1H-benzotriazole hydrate are dissolved in 35 ml of dimethylformamide. After addition of 1.09 g (5.7 mmol) of N-ethyl-N′-(dimethylaminopropyl)-carbodiimide hydrochloride and 990 μl (5.7 mmol) of ethyl-diisopropylamine, the mixture is stirred at room temperature for 30 min. A solution of compound I.5.b (1.3 g, 2.32 mmol) in 35 ml of dimethylformamide and 408 μl (2.32 mmol) of ethyl-diisopropylamine are then added and the mixture is stirred at room temperature for a further 16 h. After concentration in vacuo and purification by flash chromatography [petroleum ether/ethyl acetate 2:1→1:1→ethyl acetate], pale yellow crystals are obtained. Yield: 1.38 g (75.3%) [TLC (ethyl acetate): R_{f}=0.53; m.p.=125° C (dec.)].

I.5) 7-Ethyl-20-O-(lysyl-alanyl)-camptothecin, di-trifluoroacetate

A suspension of the above compound (1.18 g, 1.5 mmol) in dichloromethane (50 ml) is treated with anhydrous trifluoroacetic acid (5 ml) and the resulting solution is stirred at room temperature for 1 h. After concentration to a small volume in vacuo, the product is precipitated by addition of diethyl ether. The precipitate is filtered off and recrystallized from ethyl acetate. 862 mg (71.5%) of yellow crystals are obtained [TLC (ethyl acetate): R_{f}=0.05; m.p.=137° C. (dec.)].

### EXAMPLE I.6

### 7-{N^{ε}-[Fluorenyl-9-methoxycarbonyl]-L-lysyl-L-valyloxymethyl}-camptothecin, trifluoroacetate

I.6.a) 7-Hydroxymethyl-camptothecin

This compound is prepared according to the procedure of Miyasaka et al. (Chem. Pharm. Bull. 39 (1991) 2574).

I.6.b) 7-L-Valyloxymethyl)-camptothecin, trifluoroacetate

1 g (2.64 mmol) of 7-hydroxymethyl-camptothecin is dissolved in 100 ml of DMF and then treated with 100 mg of 4-N,N-dimethylaminopyridine and one equivalent of N-tert-butoxycarbonyl-L-valine-N-carboxy-anhydride and the suspension is stirred at room temperature for 16 h. It is concentrated and the residue is purified by flash chromatography on ethyl acetate/petroleum ether 1:1 and later 1.5:1. The purified material is taken up in 30 ml of dichloromethane and treated at 0° C. with 5 ml of trifluoroacetic acid. After stirring for 30 min, the mixture is concentrated and the amino-deblocked product is precipitated from dichloromethane/ether. The target compound is obtained in a total yield of 55%. [TLC (acetonitrile/water/glacial acetic acid 5:1:0.2) R_{f}=0.37]

I.6 7-{N^{ε}-[Fluorenyl-9-methoxycarbonyl]-L-lysyl-L-valyloxymethyl}-camptothecin, trifluoroacetate

560 mg of the conjugate from Example I.6.b are added to a solution of 560 mg (1.5 eq.) of N^{α},N^{ε}-bis-(tert-butoxycarbonyl)-L-lysine, 239 mg of N-hydroxybenzotriazole and 271 mg of N-ethyl-N′-(3-dimethylaminopropyl)-carbodiimide hydrochloride in 50 ml of dimethylformamide and the mixture is stirred at room temperature for two hours. It is concentrated, taken up in dichloromethane and extracted three times with water. After drying the organic phase, it is concentrated and purified by flash chromatography (petroleum ether/ethyl acetate 1:1≧ethyl acetate).

The product obtained is then taken up in 20 ml of dichloromethane, treated at 0° C. with 3 ml of trifluoroacetic acid and stirred at room temperature for one hour. After concentration and precipitation from dichloromethane/ether, the target compound is obtained in 62% yield. [TLC: acetonitrile/water/glacial acetic acid 5:1:0.2 R_{f}=0.62].

I.7 10,11-Methylenedioxy-20-O-{N^{ε}-[fluorenyl-9-methoxycarbonyl]-lysyl-leucyl}-camptothecin, trifluoroacetate

I.7.a) 10,11-Methylenedioxy-camptothecin

This camptothecin derivative is prepared according to Wall et al. (J. Med. Chem. 29 (1986), 2358) from the enantiomerically pure tricyclic compound having the S configuration, which can be obtained, for example, by resolution of the racemate.

I.7.b) 10,11-(Methylenedioxy)-20-O-leucyl-camptothecin, trifluoroacetate

150 mg (0.382 mmol) of 10,11-methylenedioxy-camptothecin are dissolved in 20 ml of DMF and then treated with 20 mg of 4-N,N-dimethylaminopyridine and 10 equivalents of N-tert-butoxycarbonyl-L-leucine-N-carboxy-anhydride and the suspension is stirred at 40° C. for 16 h. It is concentrated and purified by flash chromatography on ethyl acetate/petroleum ether 2:1. The purified material is taken up in 15 ml of dichloromethane and treated at 0° C. with 2 ml of trifluoroacetic acid. After stirring for 30 min, it is concentrated and the amino-deblocked product is precipitated from dichloromethane/methanol using ether. The target compound is obtained in a total yield of 35%.

I.7.c) 10,11-Methylenedioxy-20-O-{N^{ε}-[fluorenyl-9-methoxycarbonyl]-lysyl-leucyl}-camptothecin, trifluoroacetate

The conjugate from Example I.7.b is linked to N^{α}-(tert-butoxycarbonyl)-N^{ε}-(fluorenyl-9-methoxycarbonyl-)-lysine according to the standard procedure (see Example I.1.a) and then deblocked on the α-amino function by action of trifluoroacetic acid. Yield: 69% over 2 stages. [TLC: acetonitrile/water 10:1 R_{f}=0.4].

### EXAMPLE I.8

### 20-O-(Lysyl-aspartyl)-camptothecin, di-hydrobromide

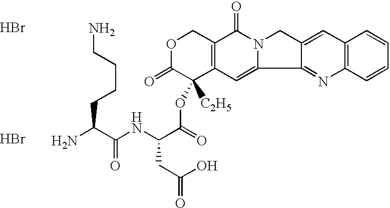

I.8.a) 20-O-[N-(tert-Butoxycarbonyl)-aspartyl-(γ-benzyl ester)]-camptothecin

A suspension of 5.23 g (15.0 mmol) of 20(S)-camptothecin in 400 ml of absolute dimethylformamide is treated with stirring with 10.45 g (30.0 mmol) of N-(tert-butoxycarbonyl)-aspartic acid (γ-benzyl ester)-N-carboxylic anhydride and 367 mg (3.0 mmol) of 4-(N,N-dimethylamino)-pyridine. After stirring at 60° C. for 8 h, a further 5.23 g (15.0 mmol) of N-(tert-butoxycarbonyl)-aspartic acid-(γ-benzyl ester)-N-carboxylic anhydride and 183.5 mg (1.5 mmol) of 4-(N,N-dimethylamino)-pyridine are added and the mixture is stirred at room temperature for three days. It is then concentrated in vacuo and the residue is purified by flash chromatography [petroleum ether/ethyl acetate 1:2]. 2.3 g (23.4%) of orange-yellow crystals are obtained [TLC (ethyl acetate): R_{f}=0.59; m.p.=130° C. (dec.)].

I.8.b) 20-O-Aspartyl-(γ-benzyl ester)-camptothecin, trifluoroacetate

A solution of compound I.8.a (2.22 g, 3.4 mmol) in a mixture of 70 ml of dichloromethane and 7 ml of anhydrous trifluoroacetic acid is stirred at room temperature for 90 min. After concentration in vacuo to a small volume, the product is precipitated with diethyl ether and washed thoroughly with diethyl ether. 1.08 g (72.3%) of beige crystals are obtained [TLC (ethyl acetate): R_{f}=0.14; m.p.=216° C. (dec.)].

I.8.c) 20-O-[N^{α},N^{ε}-di-(tert-Butoxycarbonyl)-lysyl-aspartyl-(γ-benzyl ester)]-camptothecin

433 mg (1.25 mmol) of N,N-di-(tert-butoxycarbonyl)-lysine and 338 mg (2.50 mmol) of 1-hydroxy-1H-benzotriazole hydrate are dissolved in 15 ml of dimethylformamide. After addition of. 360 mg (1.88 mmol) of N-ethyl-N′-(dimethylaminopropyl)-carbodiimide hydrochloride and 500 μ1 (3.0 mmol) of ethyl-diisopropylamine, the mixture is stirred at room temperature for 15 min. A solution of compound I.8.b (500.7 mg, 0.75 mmol) in 15 ml of dimethylformamide and 200 μl (1.13 mmol) of ethyl-diisopropylamine are then added and the mixture is stirred at room temperature for a further 16 h. After concentration in vacuo, the residue is taken up in dichloromethane and the solution is washed once with water. It is dried over MgSO₄ and the residue which remains after concentration in vacuo is purified by flash chromatography [petroleum ether/ethyl acetate 1:2] to give beige crystals. Yield: 473.8 mg (70.5%) [TLC (ethyl acetate): R_{f}=0.42; m.p.=99° C. (dec.)].

I.8) 20-O-(Lysyl-aspartyl)-camptothecin, di-hydrobromide

A solution of the above compound (462 mg, 0.52 mmol) in dichloromethane (25 ml) is treated with a 33% strength solution of hydrogen bromide in glacial acetic acid (5 ml) and the suspension resulting after a few minutes is stirred at room temperature for 1 h. The precipitated product is decanted off and the residue is washed thoroughly with diethyl ether. For purification, after dissolution in warm ethanol the product is reprecipitated by addition of diethyl ether. 391 mg (100%) of yellow crystals are obtained [TLC (acetonitrile/water 5:1): R_{f}=0.05; m.p.=225° C. (dec.)].

### EXAMPLE I.9

### 20-O-(Lysyl-seryl)-camptothecin, di-hydrobromide

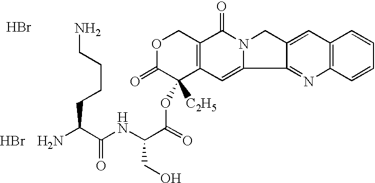

I.9.a) 20-O-[O-Benzyl-N-(tert-butoxycarbonyl)-seryl]-camptothecin

A suspension of 5.23 g (15.0 mmol) of 20(S)-camptothecin in 400 ml of absolute dimethylformamide is treated with stirring with 9.64 g (30.0 mmol) of O-benzyl-N-(tert-butoxycarbonyl)-serine-N-carboxylic anhydride and 367 mg (3.0 mmol) of 4-(N,N-dimethylamino)-pyridine. After stirring at 60° C. for 8 h, a further 4.82 g (15.0 mmol) of O-benzyl-N-(tert-butoxycarbonyl)-serine-N-carboxylic anhydride and 183.5 mg (1.5 mmol) of 4-(N,N-dimethylamino)-pyridine are added and the mixture is stirred at room temperature for three days. The mixture is then filtered, the filtrate is concentrated in vacuo and the residue is purified by flash chromatography [petroleum ether/ethyl acetate 2:1→1:1→1:2]. 6.66 g (70.9%) of a yellow foam are obtained [TLC (acetonitrile/ethyl acetate 1:1): R_{f}=0.66; FAB-MS: m/e=626 (M+H⁺)].

I.9.b) 20-O-[O-Benzyl-seryl]-camptothecin, trifluoroacetate

A solution of compound I.9.a (2.5 g, 4.0 mmol) in a mixture of 20 ml of dichloromethane and 4 ml of anhydrous trifluoroacetic acid is stirred at room temperature for 1 h. After concentration in vacuo to a small volume, the product is precipitated with diethyl ether and washed thoroughly with diethyl ether. 2.51 g (98.1%) of yellow crystals are obtained [TLC (acetonitrile/ethyl acetate 1:1): R_{f}=0.17; m.p.=198° C. (dec.)].

I.9.c) 20-O-[N^{α},N^{ε}-di-(tert-Butoxycarbonyl)-lysyl-(O-benzyl)-seryl]-camptothecin

1.73 g (5.0 mmol) of N,N-di-(tert-butoxycarbonyl)-lysine and 1.35 g (10 mmol) of 1-hydroxy-1H-benzotriazole hydrate are dissolved in 50 ml of dimethylformamide. After addition of 1.44 g (7.5 mmol) of N-ethyl-N′-(dimethylaminopropyl)-carbodiimide hydrochloride and 2.0 ml (12 mmol) of ethyl-diisopropylamine, the mixture is stirred at room temperature for 15 min. A solution of compound I.9.b (1.92 g, 3.0 mmol) in 50 ml of dimethylformamide and 790 μl (4.5 mmol) of ethyl-diisopropylamine are then added and the mixture is stirred at room temperature for a further 16 h. After concentration in vacuo, the residue is purified by flash chromatography [petroleum ether/ethyl acetate 3:1→1:1→1:3] to give yellow crystals. Yield: 2.32 g (89.1%) [TLC (ethyl acetate): R_{f}=0.45; m.p.=130° C. (dec.)].

I.9) 20-O-(Lysyl-seryl)-camptothecin, di-hydrobromide

A solution of the above compound (2.13 g, 2.46 mmol) in dichloromethane (120 ml) is treated with a 33% strength solution of hydrogen bromide in acetic acid (25 ml) and the suspension resulting after a few minutes is stirred at room temperature for 1 h. The precipitated product is decanted off and the residue is washed thoroughly with diethyl ether. For purification, after dissolution in dichloromethane/methanol 1:1 the product is reprecipitated by addition of diethyl ether. 1.78 g (100%) of yellow crystals are obtained [TLC (acetonitrile/water 5:1): R_{f}=0.05].

### EXAMPLE I.10

### 7-Ethyl-20-O-[N^{ε}-(fluorenyl-9-methoxycarbonyl)-lysyl-valyl]-camptothecin, trifluoroacetate

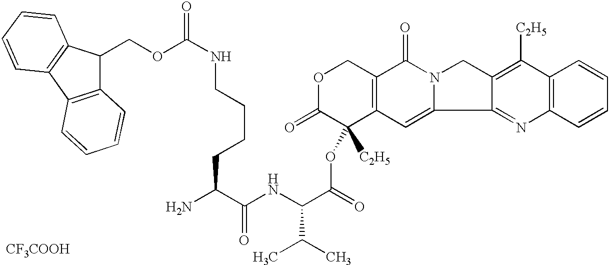

I.10.a) 20-O-[N-(tert-Butoxycarbonyl)-valyl]-7-ethyl-camptothecin

Using the process described in I.5.a, the compound is prepared from 1.88 g (5.0 mmol) of 20(S)-7-ethyl-camptothecin (S. Sawada et al., Chem. Pharm. Bull. 39 (1991) 1446-1454) and 2.43 g (10.0 mmol) of N-(tert-butoxycarbonyl)-valine-N-carboxylic anhydride. 1.46 g (51%) of beige crystals are obtained [TLC (acetonitrile): R_{f}=0.86; m.p.=224-227° C. (dec.); FAB-MS: m/e=576 (M+H⁺)].

I.10.b) 7-Ethyl-20-O-valyl-camptothecin, trifluoroacetate

The N-(tert-butoxycarbonyl) group is removed from compound I.10.a (1.44 g, 2.5 mmol) as described under I.5.b. 626 mg (43%) of yellow crystals are obtained [TLC (acetonitrile): R_{f}=0.45; m.p.=160° C. (dec.)].

I.10.c) 20-O-[N^{α}-(tert-Butoxycarbonyl)-N^{ε}-(fluorenyl-9-methoxycarbonyl)-lysyl-valyl]-7-ethyl-camptothecin

In analogy to I.5.c, 797 mg (1.7 mmol) of N^{α}-(tert-butoxycarbonyl)-N^{ε}-(fluorenyl-9-methoxycarbonyl)-lysine are reacted with compound I.10.b (590 mg, 1.0 mmol). After concentration in vacuo and purification by flash chromatography [petroleum ether/ethyl acetate 1:2], beige crystals are obtained. Yield: 287 mg (31%) [TLC (ethyl acetate): R_{f}=0.50; m.p.=172° C. (dec.)].

I.10) 7-Ethyl-20-O-[N^{ε}-(fluorenyl-9-methoxycarbonyl)-lysyl-valyl]-camptothecin, trifluoroacetate

The above compound (277.8 mg, 0.3 mmol) is deprotected as described with trifluoroacetic acid in dichloromethane. 209 mg (74%) of yellow crystals are obtained [TLC (ethyl acetate): R_{f}=0.06; m.p.=199° C. (dec.)].

### EXAMPLE I.11

### 7-Ethyl-20-O-[N^{ε}-(fluorenyl-9-methoxycarbonyl)-lysyl-valyl]-10-hydroxy-camptothecin, trifluoroacetate

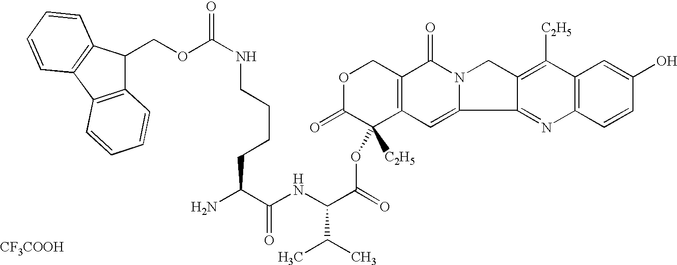

I.11.a) 20-O-[N-(tert-Butoxycarbonyl)-valyl]-7-ethyl-10-hydroxy-camptothecin

Using the process described in I.5.a, the compound is prepared from 392.4 mg (1.0 mmol) of 20(S)-7-ethyl-10-hydroxy-camptothecin (S. Sawada et al., Chem. Pharm. Bull. 39 (1991) 3183-3188) and a total of 2.43 g (10.0 mmol) of N-(tert-butoxycarbonyl)-valine-N-carboxylic anhydride in the course of 6 days. After flash chromatography [petroleum ether/ethyl acetate 5:1→2:1→1:1], 353 mg (45%) of pale yellow crystals are obtained [TLC (acetonitrile/ethyl acetate 1:1): R_{f}=0.63; m.p.=95-97° C.].

I.11.b) 7-Ethyl-10-hydroxy-20-O-valyl-camptothecin, trifluoroacetate

The N-(tert-butoxycarbonyl) group is removed from compound I.11.a (340 mg, 0.43 mmol) as described under I.5.b. 255 mg (98%) of yellow crystals are obtained [TLC (acetonitrile/ethyl acetate 1:1): R_{f}=0.04; m.p.=189° C. (dec.)].

I.11.c) 20-O-[N^{α}-(tert-Butoxycarbonyl)-N-(fluorenyl-9-methoxycarbonyl)-lysyl-valyl]-7-ethyl-10-hydroxy-camptothecin

In analogy to I.5.c, 562.3 mg (1.2 mmol) of N^{α}-(tert-butoxycarbonyl)-N^{ε}-(fluorenyl-9-methoxycarbonyl)-lysine are reacted with compound I.11.b (242.2 mg, 0.4 mmol). After concentration in vacuo and purification by flash chromatography [petroleum ether/ethyl acetate 5:1→3:1→1:1], yellow crystals are obtained. Yield: 251 mg (67%) [TLC (acetonitrile/ethyl acetate 1:1): R_{f}=0.68; m.p.=163° C. (dec.)].

I.11) 7-Ethyl-20-O-[N^{ε}-(fluorenyl-9-methoxycarbonyl)-lysyl-valyl]-10-hydroxy-camptothecin, trifluoroacetate

The above compound (244.9 mg, 0.26 mmol) is deprotected as described using trifluoroacetic acid in dichloromethane. 115 mg (46%) of yellow crystals are obtained [TLC (acetonitrile/ethyl acetate 1:1): R_{f}=0.05; m.p.=196° C. (dec.)].

### EXAMPLES 1.1-1.3

### Conjugates of Batracylin with an Amino Acid; General Formula

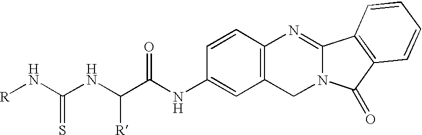

1.1) N-[N-(4-Hydroxy-phenylamino-thiocarbonyl)-D-alanyl]-batracylin

Starting material: N-(D-alanyl)-batracylin

Yield: 76% [TLC(ethyl acetate/glacial acetic acid 100:1): R_{f}=0.53; m.p.: 185° C.]

1.2) N-[N^{α}-(4-Hydroxy-phenylamino-thiocarbonyl)-lysyl]-batracylin

Starting material: N-[N^{ε}-(Fluorenyl-9-methoxycarbonyl)-lysyl]-batracylin, trifluoroacetate

Yield: 68% over 2 stages [TLC (dichloromethane/methanol 5:1): R_{f}=0.31; m.p.: 162° C. (dec.)]

1.3) N-[N^{ε}-(4-Hydroxy-phenylamino-thiocarbonyl)-lysyl]-batracylin

Starting material: N-[N^{α}-(tert-Butoxycarbonyl)-lysyl]-batracylin

Yield: 71% over 2 stages [TLC (dichloromethane/methanol 5:1): R_{f}=0.30; m.p.: 162° C. (dec.)]

### EXAMPLES 1.4-1.8

### Conjugates of Batracylin with Two Amino Acids; General Formula

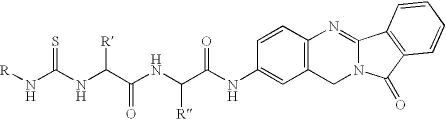

1.4) N-[N^{α}-(4-Hydroxy-phenylamino-thiocarbonyl)-lysyl-D-alanyl]-batracylin

Starting material: N-[N^{ε}-(Fluorenyl-9-methoxycarbonyl)-lysyl-D-alanyl]-batracylin, trifluoroacetate,

Yield: 70% over 2 stages [TLC (acetonitrile/water/glacial acetic acid 5:1:0.2): R_{f}=0.36]

1.5) N-[N-(4-Hydroxy-phenylamino-thiocarbonyl)-seryl-D-alanyl]-batracylin

Starting material: N-(Seryl-D-alanyl)-batracylin, trifluoroacetate

Yield: 45% [TLC (dichloromethane/methanol/ammonia 17% strength 15:2:0.2): R_{f}=0.32]

1.6) N-[N-(4-Hydroxy-phenylamino-thiocarbonyl)-glutamyl-D-alanyl]-batracylin

Starting material: N-(Glutamyl-D-alanyl)-batracylin

Yield: 70% [TLC (dichloromethane/methanol/ammonia 17% strength 15:8:0.8): R_{f}=0.68]

1.7) N-[N^{α},N^{ε}-bis-(4-Hydroxy-phenylamino-thiocarbonyl)-lysyl-seryl]-batracylin

Starting material: N-(Lysyl-seryl)-batracylin, di-trifluoroacetate

Yield: 46% [TLC (dichloromethane/methanol/ammonia 17% strength 15:3:0.2): R_{f}=0.24; m.p.: 155-157° C. (dec.)]

1.8) N-{N⁶⁰ -[N^{α},N^{ε}-bis-(4-Hydroxy-phenylamino-thiocarbonyl)-lysyl]-α,β-diaminopropionyl}-batracylin

Starting material: N-[N^{α}-Lysyl-N^{β}-(fluorenyl-9-methoxycarbonyl)-α,β-diamino-propionyl]-batracylin, di-trifluoroacetate

Yield: 39% over 2 stages [TLC (acetonitrile/water/glacial acetic acid 5:1:0.2): R_{f}=0.54]

### EXAMPLES 2.1-2.10

### Conjugates of Quinolone-a with an Amino Acid; General Formula

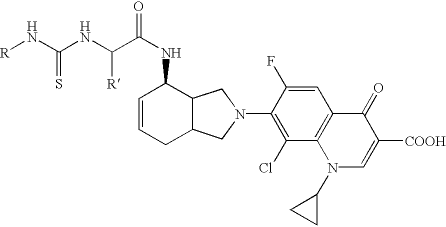

2.1) N-[N-(4-Hydroxy-phenylamino-thiocarbonyl)-alanyl]-quinolone-a

Starting material: N-(Alanyl)-quinolone-a, trifluoroacetate

Yield: 48% [TLC (acetonitrile/water 10:1): R_{f}=0.55]

2.2) N-[N-(4-Hydroxy-phenylamino-thiocarbonyl)-D-alanyl]-quinolone-a

Starting material: N-(D-Alanyl)-quinolone-a, trifluoroacetate

Yield: 61% [TLC (dichloromethane/methanol/glacial acetic acid 90:10:1): R_{f}=0.38]

2.3) N-[N^{α}-(4-Hydroxy-phenylamino-thiocarbonyl)-α,γ-diaminobutyryl]-quinolone-a, hydrochloride

Starting material: N-[^{γ}-(Fluorenyl-9-methoxycarbonyl)-α,γ-diaminobutyryl]-quinolone-a, trifluoroacetate

Salt-free precursor: 60% over 2 stages [TLC (dichloromethane/methanol/ammonia 17% strength 10:10:3): R_{f}=0.51; m.p.: 221° C. (dec.)]

Hydrochloride: The compound is suspended in water and the pH is adjusted to 2-3 using 0.1 N hydrochloric acid. After filtration, the filtrate is lyophilized.

2.4) N-[N^{α}-(4-Hydroxy-phenylamino-thiocarbonyl)-lysyl]-quinolone-a, hydrochloride

Starting material: N-[N^{ε}-(Fluorenyl-9-methoxycarbonyl)-lysyl]-quinolone-a, trifluoroacetate

Yield: 74% over 2 stages [TLC (acetonitrile/water/glacial acetic acid 5:1:0.2): R_{f}=0.33]

2.5) N-[N^{α},N^{ε}-bis-(4-Hydroxy-phenylamino-thiocarbonyl)-D-lysyl]-quinolone-a

Starting material: N-(D-Lysyl)-quinolone-a, di-trifluoroacetate

Yield: 59% [TLC (acetonitrile/water/glacial acetic acid 5:1:0.2): R_{f}=0.33; m.p.: 186° C.]

2.6) N-[N^{α}-(4-Hydroxy-phenylamino-thiocarbonyl)-ornithyl]-quinolone-a, hydrochloride

Starting material: N-[N^{δ}-(Fluorenyl-9-methoxycarbonyl)-ornithyl]-quinolone-a, trifluoroacetate

Salt-free precursor: 47% over 2 stages [TLC (dichloromethane/methanol/ammonia 17% strength 10:10:3): R_{f}=0.36; m.p.: 211° C. (dec.)]

Hydrochloride: The compound is suspended in water and the pH is adjusted to 2-3 using 0.1 N hydrochloric acid. After filtration, the filtrate is lyophilized.

2.7) N-[N^{α}-(Phenylamino-thiocarbonyl)-lysyl]-quinolone-a

Starting material: N-[N^{ε}-(Fluorenyl-9-methoxycarbonyl)-lysyl]-quinolone-a, trifluoroacetate

Yield: 58% over 2 stages [TLC (acetonitrile/water/glacial acetic acid 5:1:0.2): R_{f}=0.48]

2.8) N-[N^{α}-(4-Isothiocyanato-phenylamino-thiocarbonyl)-lysyl]-quinolone-a

Starting material: N-[N^{ε}-(Fluorenyl-9-methoxycarbonyl)-lysyl]-quinolone-a, trifluoroacetate

Yield: 73% over 2 stages [TLC (acetonitrile/water/glacial acetic acid 5:1:0.2): R_{f}=0.38]

2.9) N-[N^{α}-(4-Carboxy-phenylamino-thiocarbonyl)-lysyl]-quinolone-a

Starting material: N-[N^{ε}-(Fluorenyl-9-methoxycarbonyl)-lysyl]-quinolone-a, trifluoroacetate

Yield: 62% over 2 stages [TLC (acetonitrile/water/glacial acetic acid 10:3:1.5): R_{f}=0.6]

2.10) N-[N^{α}-(Phenyl-methyl-amino-thiocarbonyl)-lysyl]-quinolone-a

Starting material: N-[N^{ε}-(Fluorenyl-9-methoxycarbonyl)-lysyl]-quinolone-a, trifluoroacetate

Yield: 59% over 2 stages [TLC (acetonitrile/water/glacial acetic acid 5:1:0.2): R_{f}=0.44]

### EXAMPLE 2.11

### Conjugates of Quinolone-a with Two Amino Acids; General Formula

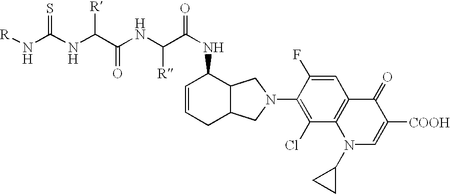

2.11) N-[N^{α}-(4-Hydroxy-phenylamino-thiocarbonyl)-lysyl-D-alanyl]-quinolone-a

Starting material: N-[N^{ε}-(Fluorenyl-9-methoxycarbonyl)-lysyl-D-alanyl]-quinolone-a trifluoroacetate

Yield: 53% over 2 stages [TLC (acetonitrile/water/glacial acetic acid 5:1:0.2) R_{f}=0.33]

### EXAMPLE 3

### Conjugates of Doxorubicin; General Formula

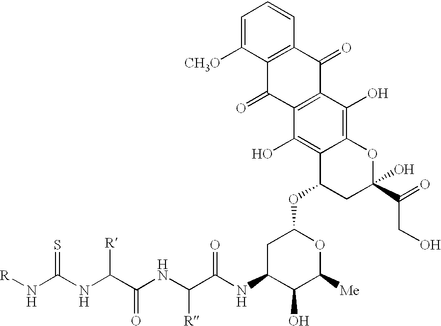

3) N-[N^{α}-(4-Hydroxy-phenylamino-thiocarbonyl)-lysyl-alanyl]-doxorubicin

Starting material: N-[N^{ε}-(Fluorenyl-9-methoxycarbonyl)-lysyl-alanyl]-doxorubicin, trifluoroacetate,

Yield: 46% over 2 stages [TLC (acetonitrile/water/glacial acetic acid 5:1:0.2): R_{f}=0.2; FAB-MS: m/e=894 (M+H)⁺]

### EXAMPLES 4.1-4.11

### Conjugates of 20(S)-Camptothecin; General Formula

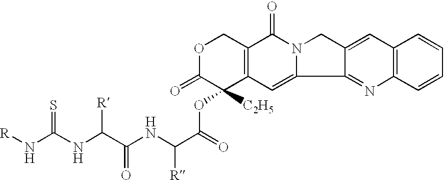

4.1) 20-O-[N^{α}-(4-Hydroxy-phenylamino-thiocarbonyl)-lysyl-alanyl]-camptothecin

Starting material: 20-O-[N^{ε}-(Fluorenyl-9-methoxycarbonyl)-lysyl-alanyl]-camptothecin, trifluoroacetate

Yield: 80% over 2 stages [TLC (acetonitrile/water/glacial acetic acid 5:1:0.2): R_{f}=0.32]

4.2) 20-O-[N^{α}-(4-Hydroxy-phenylamino-thiocarbonyl)-lysyl-leucyl]-camptothecin, hydrochloride

Starting material: 20-O-[N^{ε}-(Fluorenyl-9-methoxycarbonyl)-lysyl-leucyl]-camptothecin, trifluoroacetate

Salt-free precursor: 71% over 2 stages [TLC (acetonitrile/water/glacial acetic acid 5:1:0.2): R_{f}=0.48]

Hydrochloride: The compound is dissolved in dioxane/water and converted into the hydrochloride using one equivalent of 0.1 N hydrochloric acid. The resulting solution is then lyophilized.

4.3) 20-O-[N^{α}-(4-Hydroxy-phenylamino-thiocarbonyl)-lysyl-phenyl-alanyl]-camptothecin

Starting material: 20-O-[N^{ε}-(Fluorenyl-9-methoxycarbonyl)-lysyl-phenylalanyl]-camptothecin, trifluoroacetate

Yield: 75% over 2 stages [TLC (acetonitrile/water/glacial acetic acid 5:1:0.2): R_{f}=0.33]

4.4) 20-O-[N^{α}-(4-Hydroxy-phenylamino-thiocarbonyl)-lysyl-valyl]-camptothecin, hydrochloride

Starting material: 20-O-[N^{ε}-(Fluorenyl-9-methoxycarbonyl)-lysyl-valyl]-camptothecin, trifluoroacetate

Salt-free precursor: 68% over 2 stages [TLC (acetonitrile/water/glacial acetic acid 5:1:0.2): R_{f}=0.35; FAB-MS: m/e=727 (M+H)⁺]

Hydrochloride: The compound is dissolved in dioxane/water and converted into the hydrochloride using one equivalent of 0.1 N hydrochloric acid. The resulting solution is then lyophilized.

4.5) 20-O-[N^{α}-(4-Carboxy-phenylamino-thiocarbonyl)-lysyl-valyl]-camptothecin, hydrochloride

Starting material: 20-O-[N^{ε}-(Fluorenyl-9-methoxycarbonyl)-lysyl-valyl]-camptothecin, trifluoroacetate

Salt-free precursor: 79% over 2 stages [TLC (acetonitrile/water/glacial acetic acid 5:1:0.2): R_{f}=0.46]

Hydrochloride: The compound is dissolved in dioxane/water and converted into the hydrochloride using one equivalent of 0.1 N hydrochloric acid. The resulting solution is then lyophilized.

4.6) 20-O-[N^{α}-(4-Chloro-phenylamino-thiocarbonyl)-lysyl-valyl]-camptothecin, hydrochloride

Starting material: 20-O-[N^{ε}-(Fluorenyl-9-methoxycarbonyl)-lysyl-valyl]-camptothecin, trifluoroacetate

Salt-free precursor: 86% over 2 stages [TLC (acetonitrile/water/glacial acetic acid 10:1:0.1): R_{f}=0.24]

Hydrochloride: The compound is dissolved in dioxane/water and converted into the hydrochloride using one equivalent of 0.1 N hydrochloric acid. The resulting solution is then lyophilized.

4.7) 20-O-[N^{α}-(Phenylamino-thiocarbonyl)-lysyl-valyl]-camptothecin, hydrochloride

Starting material: 20-O-[N^{ε}-(Fluorenyl-9-methoxycarbonyl)-lysyl-valyl]-camptothecin, trifluoroacetate

Salt-free precursor: 67% over 2 stages [TLC (acetonitrile/water/glacial acetic acid 5:1:0.2): R_{f}=0.5]

Hydrochloride: The compound is dissolved in dioxane/water and converted into the hydrochloride using one equivalent of 0.1 N hydrochloric acid. The resulting solution is then lyophilized.

4.8) 20-O-[N^{α}-(Phenyl-methyl-amino-thiocarbonyl)-lysyl-valyl]-camptothecin, hydrochloride

Starting material: 20-O-[N^{ε}-(Fluorenyl-9-methoxycarbonyl)-lysyl-valyl]-camptothecin, trifluoroacetate

Salt-free precursor: 55% over 2 stages [TLC (acetonitrile/water/glacial acetic acid 5:1:0.2): R_{f}=0.5]

Hydrochloride: The compound is dissolved in dioxane/water and converted into the hydrochloride using one equivalent of 0.1 N hydrochloric acid. The resulting solution is then lyophilized.

4.9) 20-O-[N^{α},N^{ε}-bis-(4-Hydroxy-phenylamino-thiocarbonyl)-lysyl-alanyl]-camptothecin

Starting material: 20-O-(Lysyl-alanyl)-camptothecin, di-trifluoroacetate

Yield: 64% [TLC (acetonitrile/water 10:1): R_{f}=0.72]

4.10) 20-O-[N^{α},N^{ε}-bis-(4-Hydroxy-phenylamino-thiocarbonyl)-lysyl-D-alanyl]-camptothecin

Starting material: 20-O-(Lysyl-D-alanyl)-camptothecin, di-trifluoroacetate

Yield: 77% [TLC (acetonitrile/water 20:1): R_{f}=0.40; FAB-MS: m/e=850 (M+H)⁺]

4.11) 20-O-[N^{α},N^{ε}-bis-(4-Hydroxy-phenylamino-thiocarbonyl)-lysyl-phenylalanyl]-camptothecin

Starting material: 20-O-(Lysyl-phenylalanyl)-camptothecin, di-trifluoroacetate

Yield: 84% [TLC (acetonitrile/water 20:1): R_{f}=0.6]

4.12) 20-O-[N^{α}-(3-Hydroxy-phenylamino-thiocarbonyl)-lysyl-valyl]-camptothecin, hydrochloride

Starting material: 20-O-[N^{ε}-(Fluorenyl-9-methoxycarbonyl)-lysyl-valyl]-camptothecin, trifluoroacetate

Salt-free precursor: 58% over 2 stages [TLC (acetonitrile/ethyl acetate 1:1): R_{f}=0.03; m.p.=195° C. (dec.); FAB-MS: m/e=727 (M+H)⁺]

Hydrochloride: The compound is treated with water and the suspension is acidified to pH 2 using 1 N hydrochloric acid. The resulting solution is filtered through Celite and then lyophilized.

4.13) 20-O-[N^{α}-(2-Hydroxy-phenylamino-thiocarbonyl)-lysyl-valyl]-camptothecin, hydrochloride

Starting material: 20-O-[N^{ε}-(Fluorenyl-9-methoxycarbonyl)-lysyl-valyl]-camptothecin, trifluoroacetate

Salt-free precursor: 36% over 2 stages [TLC (acetonitrile/ethyl acetate 1:1): R_{f}=0.03; m.p.=192° C. (dec.); FAB-MS: m/e=727 (M+H)⁺]

Hydrochloride: The compound is treated with water and the suspension is acidified to pH 2 using 1 N hydrochloric acid. The resulting solution is filtered through Celite and then lyophilized.

4.14) 20-O-[N^{α}-(4-Methoxy-phenylamino-thiocarbonyl)-lysyl-valyl]-camptothecin, hydrochloride

Starting material: 20-O-[N^{ε}-(Fluorenyl-9-methoxycarbonyl)-lysyl-valyl]-camptothecin, trifluoroacetate

Salt-free precursor: 54% over 2 stages [TLC (acetonitrile/ethyl acetate 1:1): R_{f}=0.06; m.p.=195° C. (dec.); FAB-MS: m/e=741 (M+H)⁺]

Hydrochloride: The compound is treated with water and the suspension is acidified to pH 2 using 1 N hydrochloric acid. The resulting solution is filtered through Celite and then lyophilized.

4.15) 20-O-[N^{α}-(3-Methoxy-phenylamino-thiocarbonyl)-lysyl-valyl]-camptothecin, hydrochloride

Starting material: 20-O-[N^{ε}-(Fluorenyl-9-methoxycarbonyl)-lysyl-valyl]-camptothecin, trifluoroacetate

Salt-free precursor: 65% over 2 stages [TLC (acetonitrile/ethyl acetate 1:1): R_{f}=0.08; m.p.=197° C. (dec.); FAB-MS: m/e=741 (M+H)⁺]

Hydrochloride: The compound is treated with water and the suspension is acidified to pH 2 using 1 N hydrochloric acid. The resulting solution is filtered through Celite and then lyophilized.

4.16) 20-O-[N^{α}-(4-Nitro-phenylamino-thiocarbonyl)-lysyl-valyl]-camptothecin, hydrochloride

Starting material: 20-O-[N^{ε}-(Fluorenyl-9-methoxycarbonyl)-lysyl-valyl]-camptothecin, trifluoroacetate

Salt-free precursor: 86% over 2 stages [TLC: (acetonitrile/water/glacial acetic acid 5:1:0.2): R_{f}=0.5].

Hydrochloride: The compound is dissolved using dioxane/water and converted into the hydrochloride using one equivalent of 0.1 N hydrochloric acid. The resulting solution is then lyophilized.

4.17) 20-O-[N^{α}-(3-Nitro-phenylamino-thiocarbonyl)-lysyl-valyl]-camptothecin, hydrochloride

Starting material: 20-O-[N^{ε}-(Fluorenyl-9-methoxycarbonyl)-lysyl-valyl]-camptothecin, trifluoroacetate

Salt-free precursor: 46% over 2 stages. The Fmoc-protected intermediate is purified by flash chromatography on silica gel using dichloromethane/methanol 50:1). Deblocking is then carried out using piperidine [TLC: (acetonitrile/water/glacial acetic acid 5:1:0.2): R_{f}=0.45;

Hydrochloride: The compound is dissolved in water and converted into the hydrochloride with one equivalent of 0.1 N hydrochloric acid. The resulting solution is then lyophilized [FAB-MS: m/e=756 (M+H)⁺].

4.18) 20-O-[N^{α}-(4-Amino-phenylamino-thiocarbonyl)-lysyl-valyl]-camptothecin, hydrochloride

Starting material: 20-O-[N^{ε}-(Fluorenyl-9-methoxycarbonyl)-lysyl-valyl]-camptothecin, trifluoroacetate

mono-Fmoc-protected p-phenylene-diamine: This is prepared from phenylenediamine using 0.5 eq. of Fmoc-Cl without further addition of base. It is then converted into the mustard oil according to standard conditions.

Salt-free precursor: 46% over 2 stages. The Fmoc-protected intermediate is purified by flash chromatography on silica gel using dichloromethane/methanol 50:1). Deblocking is then carried out using piperidine. Purification is then carried out again by flash chromatography on silica gel using dichloromethane/methanol/ammonia 17% strength 15:1:0.1). [TLC: (acetonitrile/water/glacial acetic acid 5:1:0.2) R_{f}=0.45]

Hydrochloride: The compound is dissolved in dioxane/water and converted into the hydrochloride using one equivalent of 0.1 N hydrochloric acid. The resulting solution is then lyophilized [FAB-MS: m/e=726 (M+H)⁺].

4.19) 20-O-[N^{α}-(4-Hydroxy-phenylamino-thiocarbonyl)-histidyl-valyl]-camptothecin, hydrochloride

Starting material: 20-O-[Histidyl-valyl]-camptothecin, trifluoroacetate

Salt-free precursor: 81% [TLC: (acetonitrile/water 10:1 R_{f}=0.4]

Hydrochloride: The compound is dissolved in dioxane/water and converted into the hydrochloride using one equivalent of 0.1 N hydrochloric acid. The resulting solution is then lyophilized.

4.20) 20-O-[N^{α}-(4-Hydroxy-phenylamino-thiocarbonyl)-lysyl-leucyl]-camptothecin, hydrochloride

Starting material: 20-O-[N^{ε}-(Fluorenyl-9-methoxycarbonyl)-lysyl-valyl]-camptothecin, trifluoroacetate

Salt-free precursor: 71% over 2 stages [TLC (acetonitrile/waterlglacial acetic acid 5:1:0.2) R_{f}=0.45]

Hydrochloride: The compound is dissolved in dioxane/water and converted into the hydrochloride using one equivalent of 0.1 N hydrochloric acid. The resulting solution is then lyophilized.

4.21) 20-O-{[N^{α}-N^{ε}-bis-(4-Hydroxy-phenylamino-thiocarbonyl)]-lysyl-valyl}-camptothecin, hydrochloride

Starting material: 20-O-{Lysyl-[N^{ε}-(Fluorenyl-9-methoxycarbonyl)-lysyl]-valyl}-camptothecin, bis-trifluoroacetate

Salt-free precursor: 79% over 2 stages [TLC: (acetonitrile/water/glacial acetic acid 5:1:0.2) R_{f}=0.46]; [FAB-MS: m/e=1006=(M+H)⁺].

Hydrochloride: The compound is dissolved in dioxane/water and converted into the hydrochloride using one equivalent of 0.1 N hydrochloric acid. The resulting solution is then lyophilized.

4.22) 20-O-[N^{α},N^{ε}-bis-(4-Hydroxy-phenylamino-thiocarbonyl)-lysyl-aspartyl]-camptothecin, sodium salt

Starting material: 20-O-(Lysyl-aspartyl)-camptothecin, di-hydrobromide

Salt-free precursor: 50%—purification is carried out by reprecipitating a number of times from dichloromethane/methanol 1:1 using diethyl ether [TLC (acetonitrile/water 5:1): R_{f}=0.58; m.p.=192° C. (dec.); FAB-MS: m/e=894 (M+H)⁺].

Sodium salt: The compound is suspended in water and treated with one equivalent of 0.1 N sodium hydroxide solution. The resulting solution is then lyophilized.

4.23) 20-O-[N^{α},N^{ε}-bis-(4-Hydroxy-phenylamino-thiocarbonyl)-lysyl-seryl]-camptothecin

Starting material: 20-O-(Lysyl-seryl)-camptothecin, di-hydrobromide

Yield: 36%—purification is carried out by flash chromatography [petroleum ether/ethyl acetate 2:1→1:2→ethyl acetate] [TLC (acetonitrile): R_{f}=0.70; m.p.=183° C. (dec.); FAB-MS: m/e=866 (M+H)⁺].

### EXAMPLE 5

### Conjugates of 20(S)-7-ethyl-camptothecin; General Formula

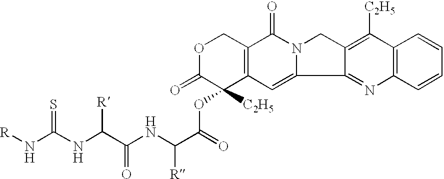

5.1) 7-Ethyl-20-O-[N^{α},N^{ε}-bis-(4-hydroxy-phenylamino-thiocarbonyl)-lysyl-alanyl]-camptothecin

Starting material: 7-Ethyl-20-O-(lysyl-alanyl)-camptothecin, di-trifluoroacetate

Yield: 27% [TLC (acetonitrile): R_{f}=0.68; m.p.=122° C. (dec.); FAB-MS: m/e=879 (M+H)⁺].

5.2) 7-Ethyl-20-O-[N^{α}-(4-hydroxy-phenylamino-thiocarbonyl)-lysyl-valyl]-camptothecin, hydrochloride

Starting material: 7-Ethyl-20-O-[N^{ε}-(fluorenyl-9-methoxycarbonyl)-lysyl-valyl]-camptothecin, trifluoroacetate

Salt-free precursor: 61% over 2 stages [beige crystals; TLC (acetonitrile/ethyl acetate 1:1): R_{f}=0.02; m.p.=220° C. (dec.); FAB-MS: m/e=755 (M+H)⁺].

Hydrochloride: The compound is treated with water and the suspension is acidified to pH 2 using 1 N hydrochloric acid. The resulting solution is filtered through Celite and then lyophilized.

### EXAMPLE 6

### Conjugates of 10,11-(methylenedioxy)-camptothecin; General Formula

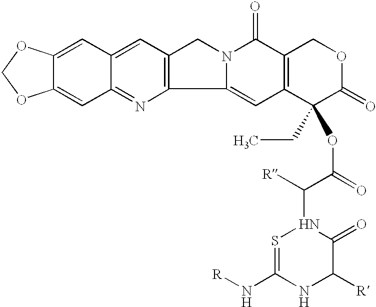

6) 10,11-(Methylenedioxy)-20-O-[N^{α}-(4-hydroxy-phenylaminothio-carbonyl)-lysyl-leucyl]-camptothecin, hydrochloride

Starting material: 10,11-(Methylenedioxy)-20-O-[N^{ε}-(fluorenyl-9-methoxy-carbonyl)-lysyl-leucyl]-camptothecin, trifluoroacetate

Salt-free precursor: 90% over 2 stages [TLC: (acetonitrile/water/glacial acetic acid 5:1:0.2) R_{f}=0.43]

Hydrochloride: The compound is dissolved in dioxane/water and converted into the hydrochloride using one equivalent of 0.1 N hydrochloric acid. The resulting solution is then lyophilized.

### EXAMPLE 7

### Conjugates of 7-hydroxymethyl-camptothecin; General Formula

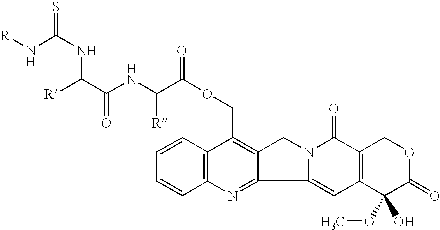

7) 7-[N^{α}-(4-Hydroxy-phenylamino-thiocarbonyl)-lysyl-valyloxymethyl]-camptothecin, hydrochloride

Starting material: 7-[N^{ε}-(Fluorenyl-9-methoxy-carbonyl)-lysyl-valyloxymethyl]-camptothecin, trifluoroacetate

Salt-free precursor: 60% over 2 stages. The Fmoc-protected intermediate is purified by flash chromatography on silica gel using dichloromethane/methanol 20:1. Deblocking is then carried out using piperidine. [TLC: (acetonitrile/water/glacial acetic acid 5:1:0.2) R_{f}=0.54]

Hydrochloride: The compound is dissolved in dioxane/water and converted into the hydrochloride using one equivalent of 0.1 N hydrochloric acid. The resulting solution is then lyophilized.

### EXAMPLE 8

### Conjugates of 20(S)-7-ethyl-10-hydroxy-camptothecin; General Formula

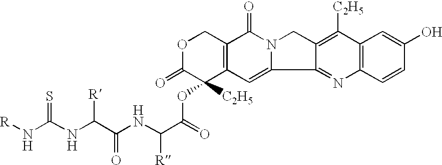

8) 7-Ethyl-10-hydroxy-20-O-[N^{α}-(4-hydroxy-phenylamino-thiocarbonyl)-lysyl-valyl]-camptothecin, hydrochloride

Starting material: 7-Ethyl-20-O-[N^{ε}-(fluorenyl-9-methoxycarbonyl)-lysyl-valyl]-10-hydroxy-camptothecin, trifluoroacetate

Salt-free precursor: 69% over 2 stages [beige crystals; TLC (acetonitrile/ethyl acetate 1:1): R_{f}=0.03; m.p.=225° C. (dec.); FAB-MS: m/e=771 (M+H)⁺]

Hydrochloride: The compound is treated with water and the suspension is acidified to pH 2 using 1 N hydrochloric acid. The resulting solution is filtered through Celite and then lyophilized.

1. Compounds of the general formula (I) 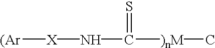
in which 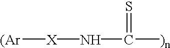
represents 1 to n′ groups 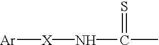
which are identical to or different from one another, where n is a number 1 to n′ and n′ corresponds to the maximum number of possible linkage sites of M,
in which
Ar represents an aryl radical having up to 10 carbon atoms, which additionally to X can optionally be mono- or polysubstituted by alkyl having up to 6 carbon atoms, alkoxy having up to 6 carbon atoms, alkoxycarbonyl having up to 6 carbon atoms, hydroxyl, carboxyl, carboxyalkyl having up to 6 carbon atoms, cyano, nitro, isocyanato, isothiocyanato, halogen, sulphonyl and/or sulphonamide,
X represents a direct single bond or alkylene having up to 6 carbon atoms,
M represents a mono-, di-, tri- or tetrapeptide, which is linked via the α-amino group and/or via amino and/or hydroxy groups of the side chains to the n groups 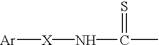
which are identical to or different from one another, where further functional groups of the peptide can optionally carry protective groups,
C represents a radical of a cytostatic or of a cytostatic derivative which is linked to m via an amino function or via an oxygen atom,
and their stereoisomers, stereoisomer mixtures and salts.

2. Compounds according to claim 1, characterized in that
Ar represents a phenyl radical which can additionally carry hydroxyl, carboxyl, isothiocyanato or halogen in the para-position to X,
and their stereoisomers, stereoisomer mixtures and salts.

3. Compounds according to one of claims 1 or 2, characterized in that
X represents a single bond or methylene,
and their stereoisomers, stereoisomer mixtures and salts.

4. Compounds according to one of claims 1, 2 or 3, characterized in that M represents a mono-, di- or tripeptide which is linked to the 1 to n groups 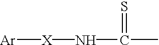
which are identical to or different from one another via the α-amino group and/or via amino and/or hydroxy groups of the side chains, where further functional groups of the peptide can optionally carry protective groups,
and their stereoisomers, stereoisomer mixtures and salts.

5. Compounds according to one of claims 1 to 4, characterized in that the peptides M consist of amino acid radicals which are derived from alanine, aspartic acid, glutamic acid, glycine, leucine, histidine, lysine, arginine, ornithine, serine, tyrosine, valine or diaminopropionic acid, it being possible for a number of amino acid radicals to be linked in peptide form both via the α-amino group and optionally via the side-chain amino functions and also via both functions,
and their stereoisomers, stereoisomer mixtures and salts.

6. Compounds according to one of claims 1 to 5, characterized in that C represents a batracylin, methotrexate, quinolone-a, etoposide, melphalan, taxol or camptothecin radical, a camptothecin derivative modified in the A ring or B ring, a daunomycin or doxorubicin radical, where C is linked to M via an amino or hydroxyl function,
and their stereoisomers, stereoisomer mixtures and salts.

7. Process for the preparation of compounds of the general formula (I) according to claim 1, characterized in that compounds of the general formula (II)
M′—C (II),
in which C has the meaning indicated in claim 1 and M′ represents a radical M defined in claim 1, which carries hydrogen atoms on the desired linkage sites and whose other potential linkage sites are blocked by protective groups,
are reacted with compounds of the general formula (III)
Ar—X—N═C═S (III)
in suitable solvents in the presence of a base to give compounds of the general formula (Ia) 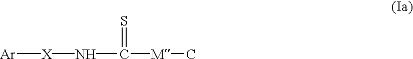
in which Ar, X and C have the meanings indicated above and M″ represents a radical M, whose further potential linkage sites are blocked by protective groups,
and in the case of the introduction of further groups 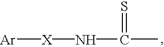
which differ from that or those initially introduced, the corresponding protective groups are optionally selectively removed from the compounds of the formula (Ia), the latter are reacted in the manner indicated above with further compounds of the general formula (III), which differ from those initially introduced, and, if appropriate, this reaction sequence is repeated to introduce further radicals 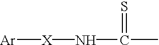
different from the radicals introduced,
and in that remaining protective groups are optionally removed,
in that, furthermore, the stereoisomers are separated, if appropriate according to customary methods, and in that, if appropriate, the compounds are converted into their salts.

8. Use of the compounds of the general formula (I) according to claim 1 for the production of medicaments.

9. Medicaments comprising compounds of the general formula (I) according to claim 1.
